(19) United States
(12) Patent Application Publication
(10) Pub. No.: US 2008/0287675 A1
(43) Pub. Date: Nov. 20, 2008

(54) CASCADE SYSTEM

(75) Inventor: Yao-En Li, Buffalo Grove, IL (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 12/120,868

(22) Filed: May 15, 2008

Related U.S. Application Data
(60) Provisional application No. 60/938,895, filed on May 18, 2007.

(51) Int. Cl. C07D 498/12

(52) U.S. Cl. 540/456

(57) ABSTRACT

A method of purifying an active pharmaceutical ingredient sufficient for administration into a human subject can include: obtaining a reaction product composition having the active pharmaceutical ingredient and impurities, wherein said active pharmaceutical ingredient is rapamycin or a rapamycin analog; introducing the reaction product composition into a first column of a chromatography system; directing a first portion of a first elutant from the first column to waste, said first portion having more impurity than active pharmaceutical ingredient; directing a second portion of the first elutant from the first column into a second column, said second portion having more active pharmaceutical ingredient than impurity; collecting factions of a second elutant from the second column that include the active pharmaceutical ingredient; and concentrating the said collected fractions to obtain a purity of the active pharmaceutical ingredient greater than 98% and with less than or about 0.95% being first and second major impurities.

### CROSS-REFERENCE TO RELATED APPLICATIONS

The present U.S. patent application claims benefit of U.S. provisional patent application having Ser. No. 60/938,895, filed May 18, 2007, entitled “Cascade System,” with Yao-En Li as inventor, which provisional application is incorporated herein by specific reference in its entirety.

### BACKGROUND OF THE INVENTION

1. The Field of the Invention

The present invention relates to systems and methods for purifying chemical compounds. More particularly, the present invention relates to novel chromatography systems and methods of use for purifying rapamycin and/or rapamycin analogs.

2. The Related Technology

Chromatography is a common laboratory technique for the purification or separation of desired products from other impurities that may exist in a composition. Typically, chromatography involves passing a mixture of the composition having the product and an elution solvent, which combined form a mobile phase, through a stationary phase, such as chromatography packing material. The result is often a highly purified product absent of most of the impurities. Chromatography may be preparative or analytical. Preparative chromatography seeks to separate the components of a mixture for further use (and is thus a form of purification). Analytical chromatography normally operates with smaller amounts of material and seeks to measure the relative proportions of analytes in a mixture. The two are not mutually exclusive because preparative chromatography techniques can be used in analyses and analytical chromatography techniques can be used to separate out products for a later use.

Column chromatography is a separation technique in which the stationary bed is within a tube. The particles of the solid stationary phase or the support coated with a liquid stationary phase may fill the whole inside volume of the tube (e.g., packed column) or be concentrated on or along the inside tube wall leaving an open, unrestricted path for the mobile phase in the middle part of the tube (e.g., open tubular column). Differences in rates of movement through the medium are calculated to different retention times of the sample.

Modern flash chromatography systems are sold as pre-packed plastic cartridges, and the solvent is pumped through the cartridge. Systems may also be linked with detectors and fraction collectors providing automation. The introduction of gradient pumps result in quicker separations and less solvent usage.

Chromatography is often used to purify a pharmaceutical ingredient for use as a drug. As such, chromatography can provide highly purified products of sufficient purity for use as drugs. This requires that the impurities be removed so as to not contaminate the pharmaceutical ingredient. However, not all chromatography methods can purify all pharmaceutical ingredients to a suitable purity. Often, a specific chromatography system and method of purification have to be utilized in order to obtain a suitable purity for a particular drug. This typically requires a substantial amount of resources, time, experiments, and costs in identifying a suitable chromatography system and method for a particular drug. Even when a suitable chromatography system and method for purifying a drug is found, the purity and/or yield is often below 100%. This allows room for improvements in most chromatography systems and methods.

Previously, chromatography systems and methods have been used to purify rapamycin and/or rapamycin analogs for pharmaceutical uses. To date, the most successful chromatography systems and methods for purifying rapamycin and/or rapamycin analogs have met minimal standards of acceptability, but still have room for improvement in purity and yield. Thus, there is a need for improved chromatography systems and methods to purify rapamycin and/or rapamycin analogs for pharmaceutical uses.

### BRIEF SUMMARY

Generally, the present invention includes systems and methods for purifying an active pharmaceutical ingredient, such as rapamycin or rapamycin analog. This includes purifying rapamycin or analog thereof from a reaction product composition or other composition.

In one embodiment, a method of purifying an active pharmaceutical ingredient sufficient for administration into a human subject can include: obtaining a reaction product composition having the active pharmaceutical ingredient and impurities, wherein said active pharmaceutical ingredient is rapamycin or a rapamycin analog; introducing the reaction product composition into a first column of a chromatography system; directing a first portion of a first elutant from the first column to waste, said first portion have more impurity than active pharmaceutical ingredient; directing a second portion of the first elutant from the first column into a second column, said second portion having more active pharmaceutical ingredient than impurity; collecting factions of a second elutant from the second column that include the active pharmaceutical ingredient; and concentrating said collected fractions to obtain a purity of the active pharmaceutical ingredient greater than 98% and with less than or about 0.95% being first and second major impurities. The method can purify either the N1 and/or N2 analogs of Formula 1. The N1 analog is a preferred rapamycin analog (e.g., zotarolimus or ABT-578) for purification.

In one embodiment, a method of purifying a rapamycin analog sufficient for administration into a human subject can include: obtaining a reaction product composition having the rapamycin analog and impurities; introducing the reaction product composition into a first column of a cascade system with an isocratic solvent system; directing a first portion of a first elutant from the first column to waste, said first portion having more impurity than active pharmaceutical ingredient; directing a second portion of the first elutant from the first column into a second column, said second portion having more active pharmaceutical ingredient than impurity; collecting factions of a second elutant from the second column that include the active pharmaceutical ingredient; and concentrating said collected fractions to obtain a purity of the active pharmaceutical ingredient greater than 98% and with less than or about 0.95% being first and second major impurities; wherein said rapamycin analog has a chemical structure of Formula 1 or derivative thereof.

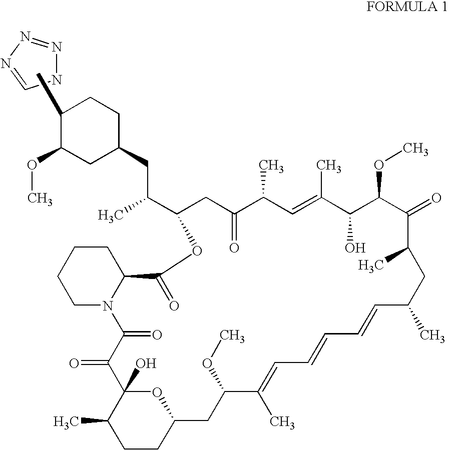

In one embodiment, a method of purifying a rapamycin analog sufficient for administration into a human subject can include: obtaining a reaction product composition having the rapamycin analog and impurities; introducing the reaction product composition into a first column of a cascade system with a step gradient solvent system; directing a first portion of a first elutant from the first column to waste, said first portion having more impurity than active pharmaceutical ingredient; directing a second portion of the first elutant from the first column into a second column, said second portion having more active pharmaceutical ingredient than impurity; collecting factions of a second elutant from the second column that include the active pharmaceutical ingredient; and concentrating said collected fractions to obtain a purity of the active pharmaceutical ingredient greater than 98% and with less than or about 0.95% being first and second major impurities; wherein said rapamycin analog has a chemical structure of Formula 1 or derivative thereof.

In one embodiment, the solvent system is an isocratic or step gradient solvent system consisting essentially of THF and heptane. However, other well known organic solvents and solvent systems may be employed.

In one embodiment, the concentrated active pharmaceutical ingredient has a purity of greater than or about 98.5% and the first major impurity is less than or about 0.85% and the second major impurity is less than or about 0.1%.

In one embodiment, the first major impurity is a retro-aldol, and the second major impurity is a N1-lutidine tetrazole adduct (N1-LTA). In one embodiment, the solvent system is a step gradient solvent system that can include the following: a first solvent introduced into the first column during a first time period between the introduction of the reaction product composition into a first column and the directing of the second portion of the first elutant from the first column into the second column; and a second solvent introduced into the first column during a second time period before, during, or after directing the second portion of the first elutant from the first column into the second column. Optionally, the first solvent includes a first ratio of THF/heptate and the second solvent includes a second ratio of THF/heptane, and wherein the first ratio is less than the second ratio or vice versa.

In one embodiment, the concentrated active pharmaceutical ingredient has a purity of greater than or about 99% and the first major impurity is less than or about 0.40% and the second major impurity is less than or about 0.1%.

In one embodiment, the step gradient solvent system includes a change from a first solvent composition to a second solvent composition at or after detecting an end portion of a peak of impurities being eluted from the first column. Alternatively, the first solvent is changed to the second solvent before the elutant from the first column includes more active pharmaceutical ingredient than impurities. In another alternative, the first solvent is changed to the second solvent when the active pharmaceutical ingredient begins to elute from the first column. In another alternative, the first solvent is changed to the second solvent substantially when a valve is switched to redirect the first elutant from waste to the second column.

In one embodiment, the first column is fluidly coupled to the second column. Alternatively, the first column is fluidly coupled to a valve that is fluidly coupled to the second column, which fluidly couples the first column to the second column. In another alternative, the first column is fluidly coupled to a valve that is fluidly coupled to the second column and a waste repository. In another alternative, the directing of the first portion of the first elutant from the first column to waste is either directly or indirectly. Directly being directed into waste instead of the second column and indirectly being directed to waste after passing through the second column. In another alternative, the directing of the second portion of the first elutant from the first column into the second column being performed without processing the second portion of the first elutant before entering the second column.

In one embodiment, the first elutant is monitored so as to determine when to direct the second portion of the first elutant to the second column. Optionally, the first elutant is monitored with UV, light absorption, light transmission, spectroscopy, mass spectroscopy, HPLC, TLC, or the like. Also, the second elutant from the second column can be similarly monitored with respect to a third column, waste, or collection.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 includes a schematic representation of an embodiment of a cascade system for chromatography separation and purification of an API.

FIG. 2A includes a schematic representation of a solvent profile for an embodiment of a isocratic chromatography separation.

FIG. 2B includes a schematic representation of a solvent profile for an embodiment of a step gradient chromatography separation.

FIG. 2C includes a schematic representation of a solvent profile for an embodiment of a continuous gradient chromatography separation.

FIG. 3 includes a graph illustrating an elution profile for an embodiment of an isocratic chromatography system that includes only a single column.

FIG. 4 includes a schematic representation of the system and process for a typical two column chromatography system with processing of API-containing elutant between columns.

FIG. 5 includes a superimposed graph that shows (1) an elution profile for an embodiment of an isocratic chromatography system that includes only a single column and (2) an elution profile for an embodiment of an isocratic cascade chromatography system that includes a cascade chromatography system.

FIG. 6A includes a graph showing an elution profile for the first column in an embodiment of an isocratic cascade chromatography system.

FIG. 6B is a schematic representation of an embodiment of an isocratic cascade chromatography system that includes the first column that produces the graph of FIG. 6A.

FIG. 6C includes a graph showing an electron profile of the entire isocratic cascade chromatography system of FIG. 6B.

FIG. 7A is a schematic representation of an embodiment of a step gradient cascade chromatography system.

FIG. 7B includes a graph showing an elution profile for the step gradient cascade chromatography system of FIG. 7A.

FIG. 8 includes a schematic diagram of an example of a chemical reaction process for preparing a rapamycin analog from rapamycin.

FIGS. 9A-9J are schematic diagraphs of the chemical structures of products of side reactions and reagent impurities obtained from the reaction shown in FIG. 8, and thereby showing reagent and product impurities to be separated from the primary product.

FIG. 10 includes a graph showing the elution profiles of the impurities and products of the reaction shown in FIG. 8, and identifies the elution of the primary product and impurities.

FIG. 11A-11B include graphs showing elution profiles for a first column (FIG. 11A) and a second column (FIG. 11B) for a two column chromatography system and method that uses the common technique of concentrating the elution product between columns.

FIG. 12 includes a graph showing an elution profile for an embodiment of an isocratic cascade chromatography system.

FIG. 13 includes a graph showing an elution profile for an embodiment of a step gradient cascade chromatography system.

FIG. 14 includes a graph showing an elution profile for an embodiment of a single column step gradient chromatography system.

### DETAILED DESCRIPTION

Generally, the present invention includes improved systems and methods for purifying chemical compounds that can be active pharmaceutical ingredients (API) for various therapeutic or prophylactic uses. Such uses can include being deposited on stents so as to form drug eluting stents that can be used to prevent and/or treat stenosis and/or restenosis. Accordingly, the present invention includes novel chromatography systems and methods of use for purifying rapamycin and/or rapamycin analogs. Accordingly, the systems and methods for purifying the API can be configured so as to produce an API that is in suitable form or can be further processed so as to be capable of being administered to a subject, such as a human patient. The improved systems and methods provide an API having increased purity than previously available.

The present invention includes systems and methods for purifying or separating the API obtained from a chemical reaction from other reaction byproducts or reagent impurities. The API can be rapamycin or a rapamycin analog, and can be purified into a suitable purity for use as a drug, on endoprostheses, deployment systems, and in methods for delivering the API from an endoprosthesis in an amount that can inhibit restenosis. More particularly, the present invention includes the use of systems and methods for separating a rapamycin analog (e.g., zotarolimus or ABT-578) from reaction byproducts and other impurities that are present in the reaction mixture. The systems and methods can purify the rapamycin analog so that it can be administered to a subject or applied to stents for use in inhibiting restenosis of the coronary artery.

### I. Introduction

In one embodiment, the present invention includes an entire chemical process that includes reaction, separation, and collection, wherein the separation is a separation method using a system as described herein. First, a reaction or series of reactions are conducted so as to produce a reaction product in the form of a composition containing the target API that is sought to be purified and collected so as to be useful as a product for use as a therapeutic. The reaction product is then loaded onto a chromatography system, such as the cascade system, isocratic cascade system, step gradient cascade system, continuous gradient cascade system, step gradient single column system, or the like. The API in a dilute solution is then collected after processing and purification by the chromatography system. The API is then collected from the solution by filtration, precipitation, concentration, drying, lyophilyzation, freeze-drying, combinations thereof, and/or other well known methods for obtaining a target API from a solution.

As used herein, a “two column” chromatography system is not considered a cascade system. In a typical two column system (which may be also be a system that includes a plurality of columns), the columns are not fluidly coupled, and the elutant from the first column is significantly processed before being directed into the second column. In a traditional two column chromatography system, the elutant of the first column is concentrated prior to being introduced into the second column, which is unfavorable as described herein. However, other processing can also occur between columns.

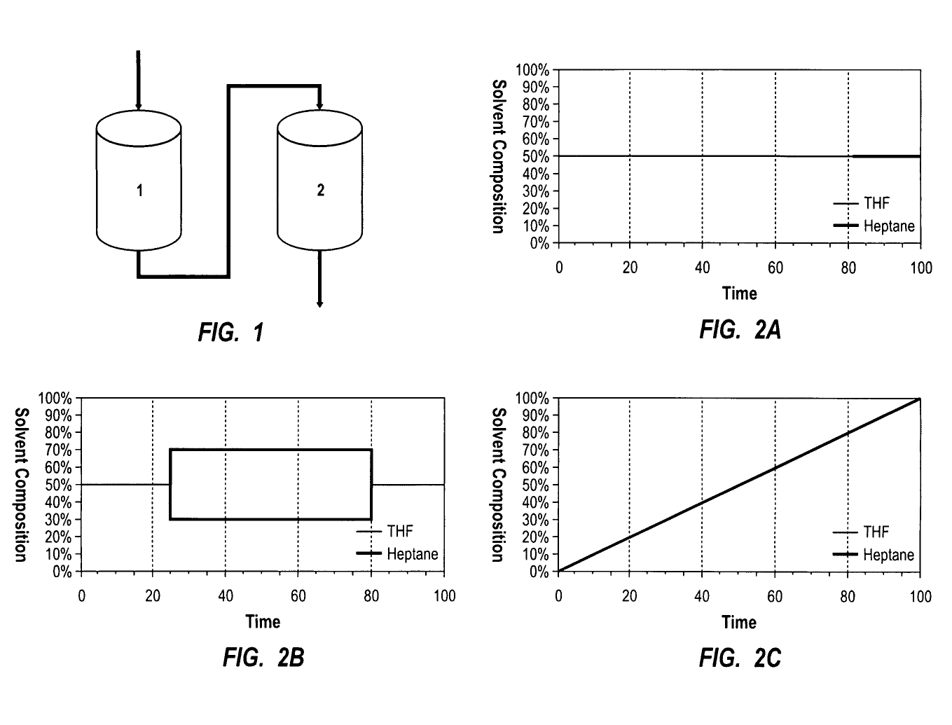

As used herein, “cascade system” is meant to refer to a chromatography system and method that includes the use of multiple columns to purify an API; however, the elutant obtained between columns is not reconstituted, concentrated, and/or otherwise processed before being introduced into the next column. This is distinguishable from other multiple column systems and methods that affirmatively reconstitute and/or concentrate (e.g., process) the API between columns. An example of a cascade system is show in FIG. 1, which illustrates a first column 1 that is continuously or fluidly coupled to a second column 2 in an uninterrupted manner however, valves or other fluid flow system devices or components can be disposed between the first column and the second column. That is, the product and elutant of column 1 flows directly into column 2, and optionally from column 2 to any additional column. Additional columns can be added after column 2 as needed or desired. Also, the elutant from column 1 can be temporarily restrained from directly flowing into column 2. For example, the elutant from column 1 can be collected before being introduced into column 2 with the caveat that the elutant is not significantly altered, condensed, concentrated, or otherwise substantially processed or manipulated before being introduced into column 2.

As used herein, “isocratic cascade system” is meant to refer to a chromatography system and method that includes the use of an isocratic solvent and multiple columns to purify an API. The solvent is isocratic so as to be a substantially constant composition having the same composition characteristics throughout the separation and purification. An example of the solvent profile for an isocratic cascade system is shown in FIG. 2A.

As used herein, “step gradient cascade system” is meant to refer to a chromatography system and method that includes the use at least a first solvent composition during a first time period and at least a second solvent composition during a second time period and multiple columns to purify an API. The step gradient cascade system is substantially similar to an isocratic cascade system except that the step gradient in the solvent at a time point. An example of the solvent profile for an step gradient cascade system is shown in FIG. 2B.

As used herein, “continuous gradient cascade system” is meant to refer to a chromatography system and method that includes the use of a solvent composition that continually changes over a constant or variant gradient for the duration of the separation or purification, and also uses multiple columns to purify an API. The continuous gradient cascade system is substantially similar to an isocratic cascade system except that the solvent continually varies over time. An example of the solvent profile for a step gradient cascade system is shown in FIG. 2C.

### II. Chromatography Systems and Methods

In one embodiment, the present invention includes a system and method that employs a cascade system for separation of the AP from other substances. The cascade system and method using the solvent systems described herein can be beneficial for at least the following: increase the yield of the API; increase the separation efficiency of the API from the impurities; reduce a significant amount of solvent usage; and reduce cycle time so that the purified API is obtained at a faster rate. The cascade system and method allows for the use of a significantly lesser amount of solvent compared to other processes that concentrate and/or reconstitute the API between columns. Also, the lack of concentrating and/or reconstituting the API significantly reduces the time to obtain purified API and significantly increases the yield. In previous systems and methods that concentrate and/or reconstitute the API between columns, a lot of solvent is used because the API is concentrated before being loaded onto the next column. With the cascade system and process, the API is only concentrated once, which is after completion of the process and the API is obtained as in a dilute solution from the final column.

In one embodiment, the present invention includes a system and method that employs an isocratic cascade system for separation of the API from other substances. An isocratic separation system and method includes a process in which the solvent ratio is kept substantially constant throughout the chromatographic separation. That is, the solvent is kept substantially the same throughout the separation. When the solvent is an initially substantially pure solvent, the isocratic method keeps the solvent substantially the same. FIG. 2A shows a schematic representation of the solvent profile in an isocratic chromatographic purification. When the solvent is a mixture of 2 or more different solvents, the solvents are initially present at an initial solvent ratio, and that solvent ratio is kept substantially the same throughout the isocratic separation process. This is different from a step gradient that changes the solvent ratio at a time point within the separation (see FIG. 2B) or a continuous gradient that substantially continually changes the solvent ratio throughout the separation (see FIG. 2C).

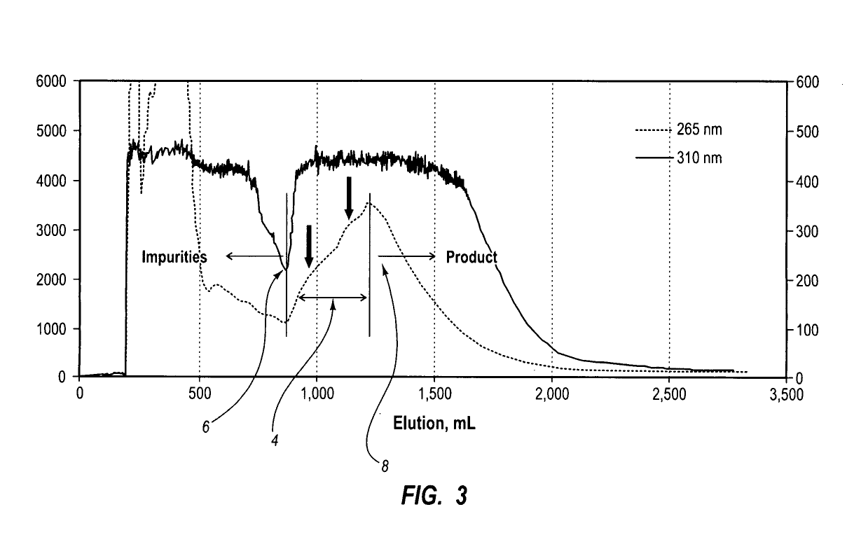

FIG. 3 is a graph illustrating a chromatography separation profile of a single column system that shows the elution products via absorbance at 265 nm and 310 nm. The column was ran as an isocratic single column with the solvent being 50% THF/heptane. The 310 nm absorbance is shown to have a first major peak that is cutoff and a second major peak between about 1000 ml to 1500 ml of elution product. As can be seen by the portion of the graph at arrow 4, the impurities co-elute with the product in the elutant between the lines that identify majority impurities 6 and majority product 8. That is, after line 8, the majority of the elutant is product. At the intermediate portion identified by arrow 4, the impurities co-elute with the produce so as to results in 5-10% loss of the API. This 5-10% loss is a result of the single column not being sufficient for separation of the API.

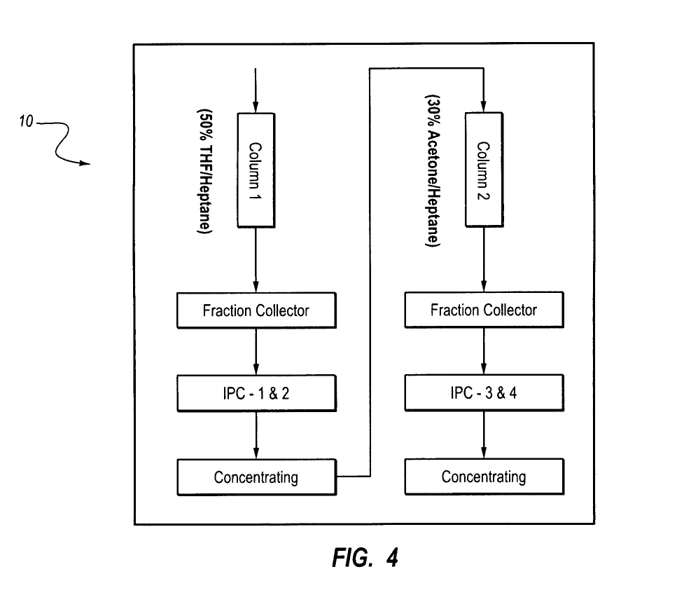

FIG. 4 includes a schematic representation of the system and process for a typical two column chromatography system 10. The two column chromatography system is shown to operate as follows: introduce reaction product solution containing target API into column 1 with a solvent that is 50% THF/heptate; process solution through column 1 so as to collect fractions; optionally analyze fractions to identify fractions that contain target API; optionally pool fractions; concentrate fractions that include API; introduce concentrated API into column 2 with a solvent that is 30% acetone and 70% heptane; process concentrated API through column 2 so as to collect fractions; optionally analyze fractions to identify fractions that contain target API; optionally pool fractions; concentrate fractions that include API; and obtain API product. Some of the problems with this traditional system are as follows: the separation of the frontal region interferes with collecting pure API; there is a long tailing API peak; the loading mixture may react with column packing material; and the concentration of API prior to second column is time consuming and can result in loss of product. For example, in the two column system, each individual fraction is usually tested for API amount and purity, which is time consuming and costly. The fractions that are sufficiently pure can be combined, which results in a significant loss of product. The intermediate concentration step is a result of a very high volume of elutant in all of the fractions. For example, the elutant volume that includes API can be about 1000 liters, which is then concentrated down to about 1 or 2 liters before being loaded onto column 2. The elutant of column 2 is collected, and the testing is performed to select the fractions containing sufficient API, and those fractions are pooled and concentrated. The process of FIG. 4 is time consuming and costly.

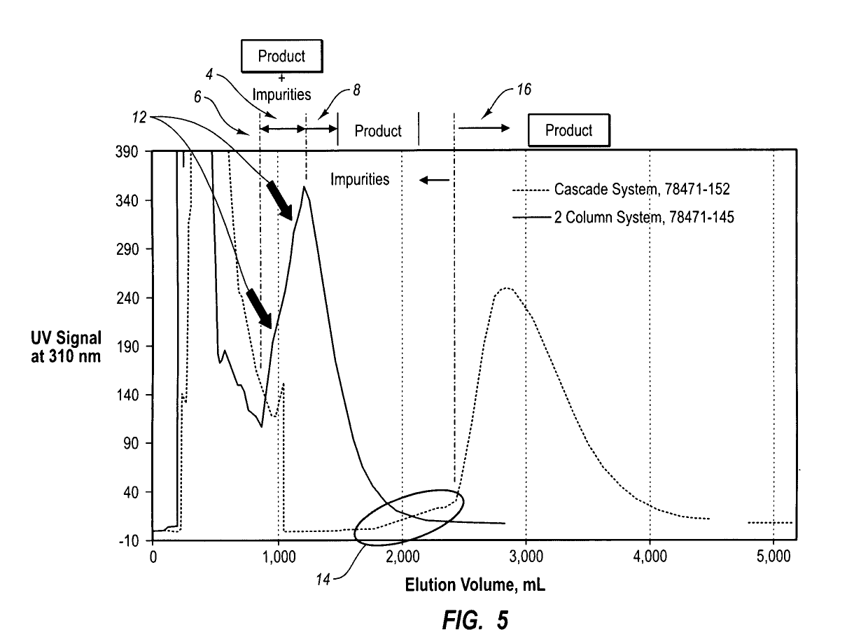

FIG. 5 is a graph that includes the chromatography profile of a single column system (solid line) as shown in FIG. 3 with the chromatography profile of a isocratic cascade system (dashed line) in accordance with the present invention. The superimposed graph shows that the single column produces a second peak that has humps identified by the arrows 12 as impurities. As such, the single column shows that the impurities co-elute with the product shown by arrow 4 in the elutant between the lines that identify majority impurities 6 and majority product 8. As such, the graph shows the API product loss of about 5-10% at the initial elution. Such a loss of product is compounded and increased by the second column in a two column system.

However, the isocratic cascade system (dashed line) shows a major initial peak where the majority of impurities are separated from the product, and then a trough where substantially only the solvent is eluted, which is followed by a slow incline (circled region 14) before the main peak that is identified as the API product. The circled region 14 of the dashed line shows the separation of impurities from the API product that are difficult to separate. As such, the cascade systems allows for impurities to be separated from the API product. The ability to separate the impurities of the circled region 14 allows for a substantial increase in both purity and yield.

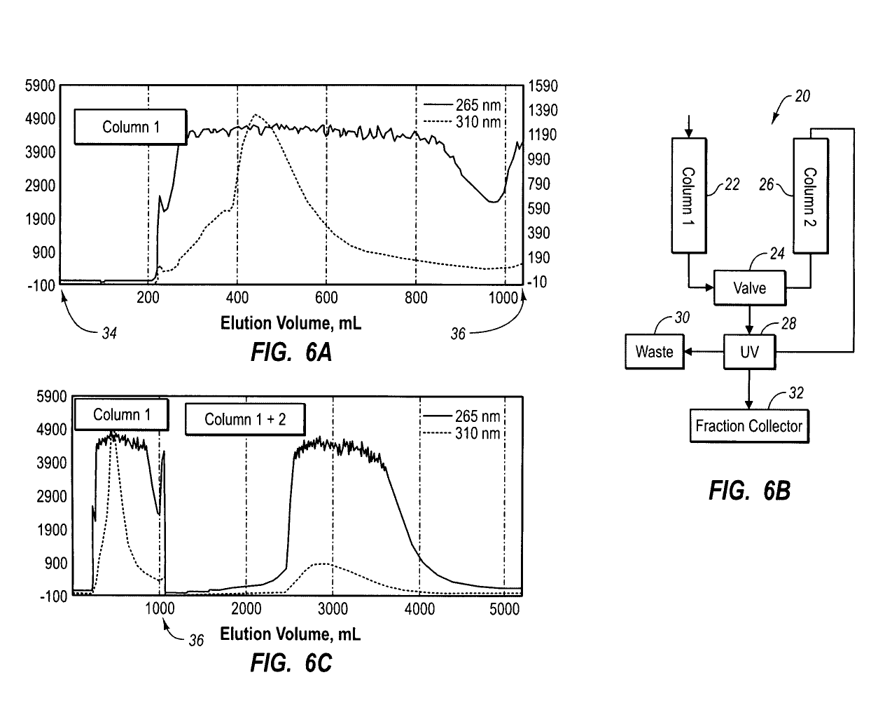

FIG. 6A shows the elution profile of a first column 22 in a cascade system 20 shown in FIG. 6B. FIG. 6B illustrates a cascade system 20 that includes a first column 22 (Column 1) that is fluidly coupled to a valve 24 that in turn is fluidly coupled to a second column 26 (Column 2). The valve 24 is additionally fluidly coupled to a UV detector 28 that is fluidly coupled to a waste depository 30 or a fraction collector 32 that collects the product. During operation, the reaction product that includes the API is introduced into the first column 22. The elutant of the first column 22 is then passed through a valve 24 that directs some of the elutant through the UV detector to determine whether or not the product includes the API in a certain amount or at a certain purity. The elutant from the first column 22 can be directed to waste 30 or collected at a fraction collector 32. In most circumstances, elutant from the first column 22 will be passed to the waste depository 30. At some point, the valve 24 is switched so as to direct the elutant from the first column 22 to the second column 26. The elutant from the second column 26 is monitored by the same or different UV detector 28 which determines which fractions of elutant are to be sent to waste 30 and which are to be collected at the fraction collector 32. For example, at an initial time point 34 the valve 24 is switched so as to direct the first column 22 elutant to waste 30. At a subsequent time point 36, the valve 24 is switched so that the first column 22 elutant is directed to the second column 26. FIG. 6A shows that when the peak of the first column 22 elutant begins to rise, the valve 24 is switched so to send the elutant to the second column 26. FIG. 6C shows the elution profile for the first column 22 and the second column 26 such that the API product has been purified by the cascade system 20. Additionally, various other well known means for chromatography operation, elutant identification, and collection can be used in accordance with the present invention.

Referring back to the cascade system elution profile graph of FIG. 5, it can be seen that at about 1000 milliliters suddenly the second peak goes down to about zero, and that is when the valve 24 is switched to the second column 26. This allows for efficient separation and purity of the target API. Accordingly, the first peak is sent to the waste and the second peak is sent to the second column. The circled area 14 of FIG. 5 shows the impurities being separated from the API by the second column. The first 1000 milliliters of elutant only go through column one, which is directed into the waste. After the first 1000 milliliters, the valve is switched and the API is thereby purified by column 1 and column 2. This essentially increases the column length to a length without having to have such a long column, which adds more difficulty and expense. A single long column may not allow for the valve to switch between waste and column 2, which may further complicate the separation.

In one embodiment, the chromatography system and method includes multiple columns in a cascade format so that the elutant from one column flows directly into the input of the next column. As such, the API is processed through multiple columns without being processed between columns, which allows the API to be obtained with higher yield in less steps. For example, the elutant from a first column is either passed to waste when there is no or extremely low API content, or to the next column when sufficient API content is present in the elutant. The elutant is not concentrated between columns, and is only concentrated after being purified/separated from impurities by all columns in the cascade format. However, other well known methods of chemical analysis or monitoring can be employed to monitor the elutant.

In one embodiment, the systems and method of the present invention utilize a UV sensor between columns to monitor the elutant from a first column. The UV detector allows for elutant that does not include API or sufficient amount of API to be passed to waste and also allows for elutant that includes API or sufficient amount of API to be passed to the next column. A UV sensor can also be placed after the exit of the last column to perform the function of API identification/characterization in order to direct the final elutant to waste or to collection for collecting API. This allows elutant with impurities to be sent to waste.

In one embodiment, the amount of API-containing composition loaded into the chromatography system is fixed. That is, an initial amount is loaded without subsequent loading. This allows for the chromatography system to remove the impurities in the volume of API by: (1) opening the valve between the columns and letting the elutant go through the UV detector and transfer elutant with impurities to the waste receptacle; (2) and after removal of initial impurities from elutant, then the valve is switched so that the elutant from the first column is processed through the second column.

In one embodiment, the entire input into the first column is processed through the entire cascade chromatography system without any elutant being sent to waste between columns or being processed. This allows the initial impurities in the elutants to be further separated by the cascade system instead of sending the initial impurities to waste. The impurities are then sent to waste after being finally eluted from the cascade system.

In one embodiment, the system and method of the invention includes a step gradient cascade chromatography system, such as where the solvent is changed at a time point during the purification/separation when the valve is switched or otherwise configured so that the elutant from the first column flows directly into the input of the next column. That is, the step gradient occurs at the time before, during, or after the elutant is directed into the second column. Alternatively, the step gradient can occur at the point where the API-containing elutant is received into the next column, which does not require a switching of the valve. An inline UV detector or other monitor can provide the time point for changing the step gradient. Also, multiple step gradients can be used. Multiple step gradients can be changed at any one or more of the following time points: after the initial impurities are separated from the API; before or at the time the initial impurities enter the subsequent column; API begins to elute from the initial column; the API begins to enter a subsequent column; after the subsequent impurities elute from the initial column; the subsequent impurities enter a subsequent column; and the like.

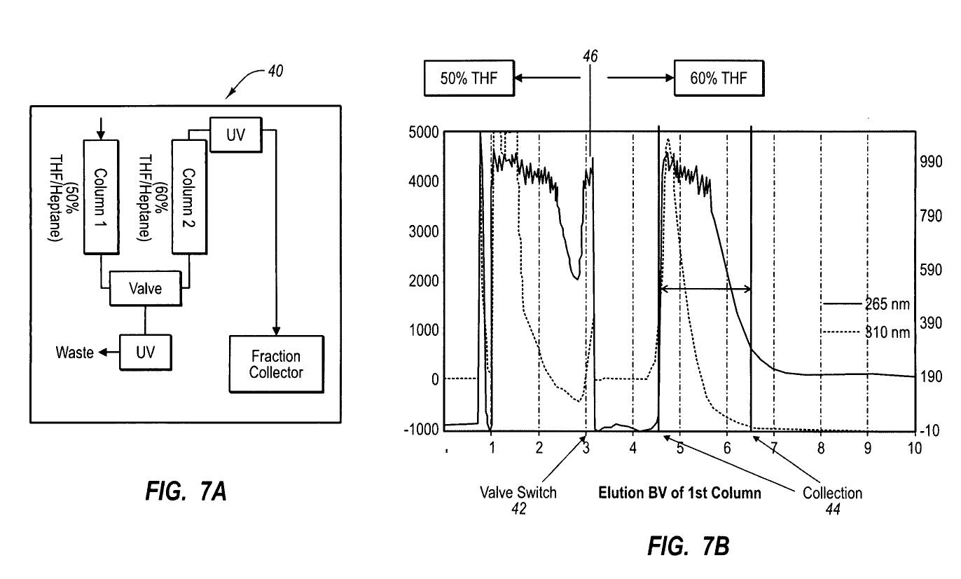

In one embodiment, at the time point that the initial first peak, as identified by UV, elutes from a first column the valve is switched so that the elutant is directed into the input of the second column. At this point the solvent can be held constant in an isocratic process or changed in a step gradient. For example, the moment a first peak is eluted, the valve is set to send the elutant to the second column and substantially simultaneously the solvent input into the first column is changed in a step gradient manner. An example of a step gradient cascade chromatography system 40 can be seen in FIG. 7A, which includes substantially all of the components shown in FIG. 6B in a slightly different arrangement. The elution profile of the step gradient cascade chromatography system 40 of FIG. 7A can be see in FIG. 7B, which shows the valve switch time point 42 and the collection period 44. Also, FIG. 7B shows that the step gradient solvent change time point 46 is substantially the same time point as the valve switch time point 42.

In one embodiment, the present invention includes a step gradient single column chromatography system. It has been shown that implementing a step gradient in a single column can improve purity of the API and reduce the total amount of impurities. For example, zotarolimus can be successfully separated from two major reaction by-products that are difficult to separate by implementing a step gradient single column chromatography technique. The step gradient can be implemented as described herein. For example, the step gradient can be implemented at any of the following time points: after the initial impurities are eluted and thereby separated from the API; after the initial impurities begin to separate from the API and before elution of the API; the API begins to elute from the column; and the like.

### III. Active Pharmaceutical Ingredient

In one embodiment, the systems and methods of the present invention can be used to purify rapamycin or a rapamycin analog. One particular rapamycin analog that can be purified with the present invention is zotarolimus, which is also referred to as ABT-578.

In one embodiment, the systems and methods described herein can purify a rapamycin analog having the structure of Formula 1, Formula 2 (42-(1-tetrazolyl)-rapamycin (more polar isomer)), Formula 3 (42-(2-tetrazolyl)-rapamycin (less polar isomer)), or a combination thereof.

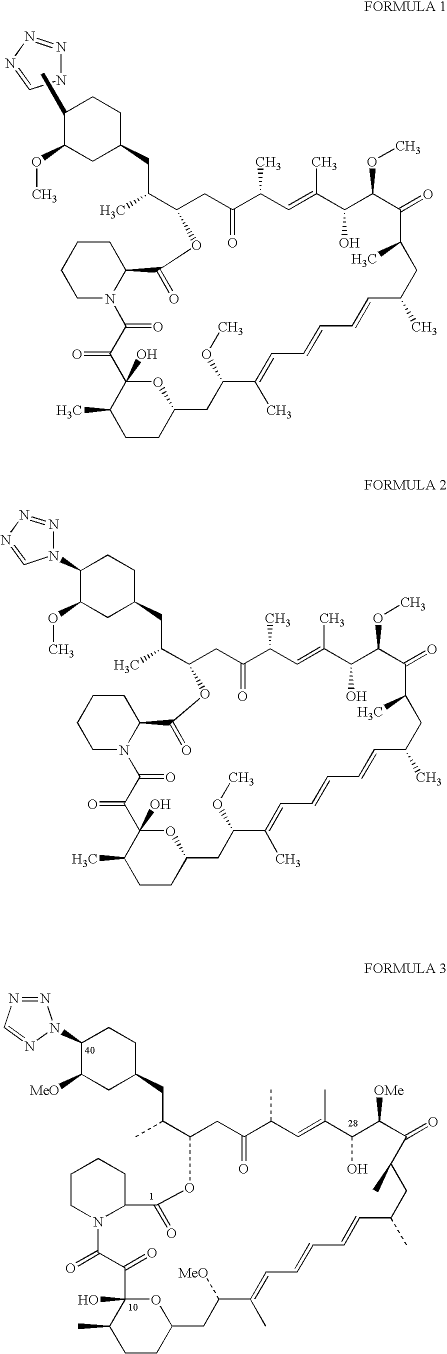

The rapamycin analog for Formula 2 can be referred to as zotarolimus or ABT-578. Additionally, the API can be any pharmaceutically acceptable salt or prodrug of the rapamycin analog. The preparation of pharmaceutically acceptable salts and/or prodrugs of bioactive agents, such as zotarolimus, are well known in the art.

In one embodiment, the rapamycin analog that is purified or separated can be a derivative of the analogs shown in Formulas 1-3. A derivative can be prepared by making minor substitutions such as hydroxylating, methylating, ethylating, or otherwise minimally altering a substituent. However, any derivative of the rapamycin analog in accordance with the present invention should have the property of inhibiting restenosis while not inhibiting cell migration as described herein.

Additionally, the rapamycin analogs of Formulas 1-3 can exist in equilibrium in solution with another analog as shown in Formulas 4A-4B. The rapamycin analog of Formula 4A can also be the corresponding analogs of Formulas 2-3 (e.g., Formula 4B). As such, the rapamycin analog of Formula 4A (and the equivalents to Formulas 2-3, such as Formula 4B) can also be purified with the systems and methods of the present invention.

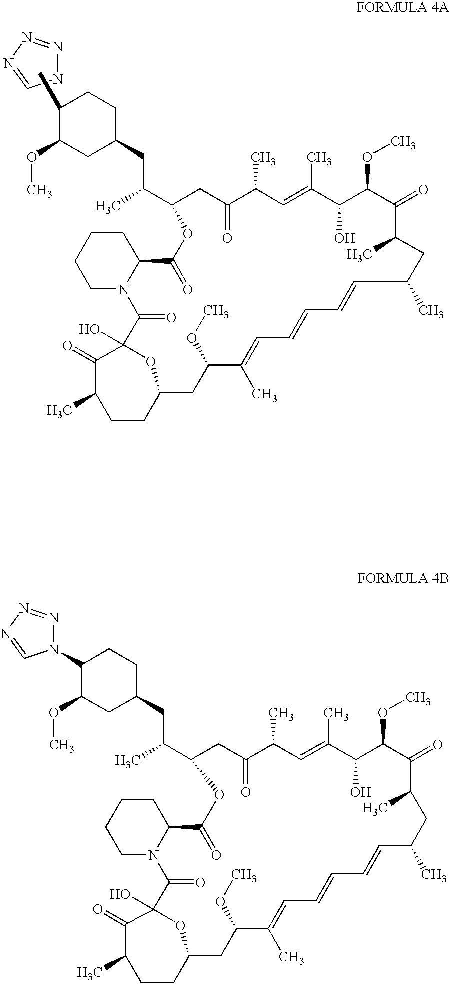

In one embodiment, the systems and methods of the present invention are utilized to purify the rapamycin analog of Formula 1 from impurities that are present in a reaction product mixture that contains the rapamycin analog.

In one embodiment, the systems and methods of the present invention are utilized to purify the rapamycin analog of Formula 2 from impurities that are present in a reaction product mixture that contains the rapamycin analog.

In one embodiment, the systems and methods of the present invention are utilized to purify the rapamycin analog of Formula 3 from impurities that are present in a reaction product mixture that contains the rapamycin analog.

In one embodiment, the systems and methods of the present invention are utilized to purify the rapamycin analog of Formula 4A and/or Formula 4B from impurities that are present in a reaction product mixture that contains the rapamycin analog.

Additional examples of rapamycin analogs that can be purified with the present invention are shown in U.S. Pre-grant Publication 2008/0085880, which is incorporated herein by reference in its entirety.

In one embodiment, the systems and methods of the present invention are utilized to purify the rapamycin analog of Formula 2 from other rapamycin analogs. That is, the other rapamycin analogs are considered impurities in such a separation. Such a separation is described in more detail below.

In one embodiment, the systems and methods of the present invention are utilized to purify the rapamycin analog of Formula 3 from other rapamycin analogs. That is, the other rapamycin analogs are considered impurities in such a separation. Such a separation is described in more detail below.

### IV. Drug Eluting Stents

In one embodiment, the systems and methods of the present invention can be used to purify an API that can be applied to an endoprosthesis. This can be used for preparing drug eluting endoprostheses, such as stents or vena cava filters.

In one embodiment, the present invention includes systems and methods for purification of rapamycin or a rapamycin analog that can be used on endoprostheses, deployment systems, and in methods for delivering rapamycin or a rapamycin analog. As such, the present invention includes systems and methods for purifying the rapamycin analog zotarolimus (e.g., ABT-578) in a manner that provides a purity sufficient for administration via an endoprosthesis. Thus, the present invention provides systems and methods for purifying zotarolimus for use in applications to inhibit restenosis without inhibiting cell migration in order to promote re-endothelialization of lesions so as to inhibit thrombosis.

Accordingly, the purified rapamycin analog in accordance with the present invention can be used in the treatment and/or prevention of hyperproliferative vascular diseases such as intimal smooth muscle cell hyperplasia, restenosis, and vascular occlusion without substantially increasing susceptibility to thrombosis. Such hyperproliferative vascular diseases may occur following biologically- or mechanically-mediated vascular injury, and can be treated or prevented by use of the drug-eluting endoprosthesis as described herein without causing thrombosis.

### EXAMPLES

### Example 1

The following types of silica were used in the studies described herein: Silica gel 5 MB 70, Fuji Silysia, Pore diameter=70 A, PSD=20-45 micro; Silica gel MB 70, Fuji Silysia, Pore diameter=70 A, PSD=20-45 micro; and Silica gel 60, D (90)=40-63 micro, Pore diameter=70 A. Additionally, other phase separation packing materials can be used in the chromatography systems and methods of the present invention.

Glass columns purchased from Knobes, Chromaflex were used in the studies described herein. However, it should be recognized that any suitable column can be used for in the chromatography systems and methods of the present invention.

Either a dry or slurry packed column can be used. Slurry packing procedure: Prepare a 1 Liter premixed 50% THF and heptane solution. Mix 250 grams of silica with about 1 liter of premixed 50% THF and heptane solution in a reactor. Transfer the contents to the glass column. Pump the premixed 50% THF and heptane into the column until there is no air bubbles detected in the effluent stream. Dry packing procedure: Weigh 250 grams of silica and transfer it to the glass column. After that, pump the premixed 50% THF and heptane solution into the column until there is no air bubbles detected in the effluent stream.

The studies described herein were conducted with Akta Purifier, an automated chromatographic unit by GE Health. However, it should be recognized that any suitable chromatographic unit can be used for in the chromatography systems and methods of the present invention.

### Example 2

The rapamycin analog was synthesized. Briefly, 3.5 gram of rapamycin is added to a pre-dried reactor, followed by the addition of 14 grams of pre-dried dichloromethane to the same reactor. The solution is stirred until the solids dissolve. 0.82 grams of 2,6-Lutidine is added, and then the reaction mixture is cooled to −30° C. 1.35 grams of trifluoromethanesulfonic anhydride is slowly added while maintaining the internal temperature at NMT −30° C. The reaction mixture is maintained at −30° C. and stirred for at least 15 minutes. Then, 0.67 grams of 1H-tetrazole is added followed by 2.48 grams of N,N-diisopropylethyl amine. The resulting reaction mixture is vigorously stirred at 25° C. for 6 hrs. The solution is ready for loading onto the chromatography column for separation. There are two major isomers in the reaction mixture: N1 (i.e., Formula 2) and N2 (i.e., Formula 3) isomers. The desired isomer is N1. The reaction schematic diagram can be see in FIG. 8.

### Example 3

The reaction product of Example 2 is separated from impurities generated by the reaction. Once the column is packed and conditioned as described, the reaction mixture is loaded onto the column. The effluent is collected in an automated fraction collector. Each fraction is then analyzed in TLC plates for product content. Based upon the TLC readings, composite of fractions with products are collected and analyzed by HPLC method. UV analysis can also be used.

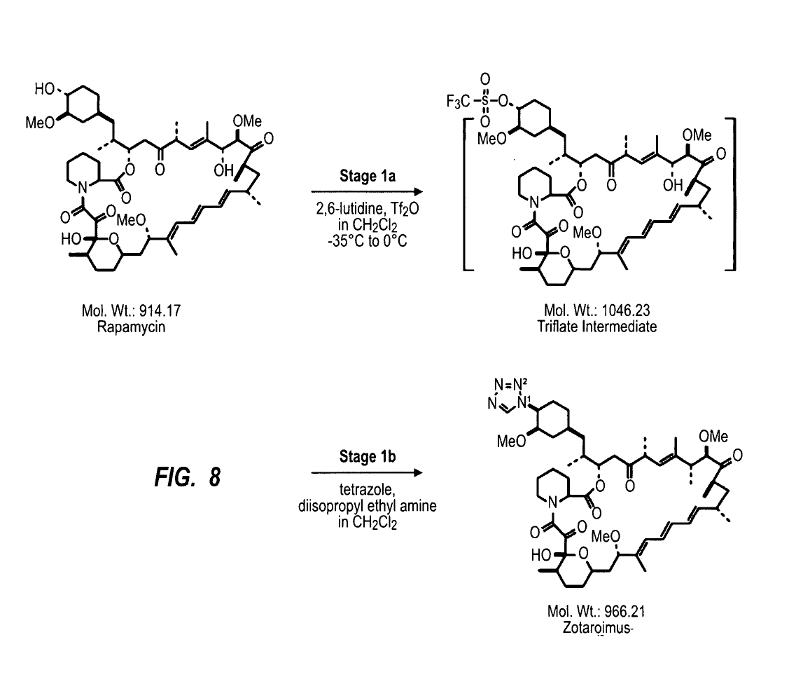

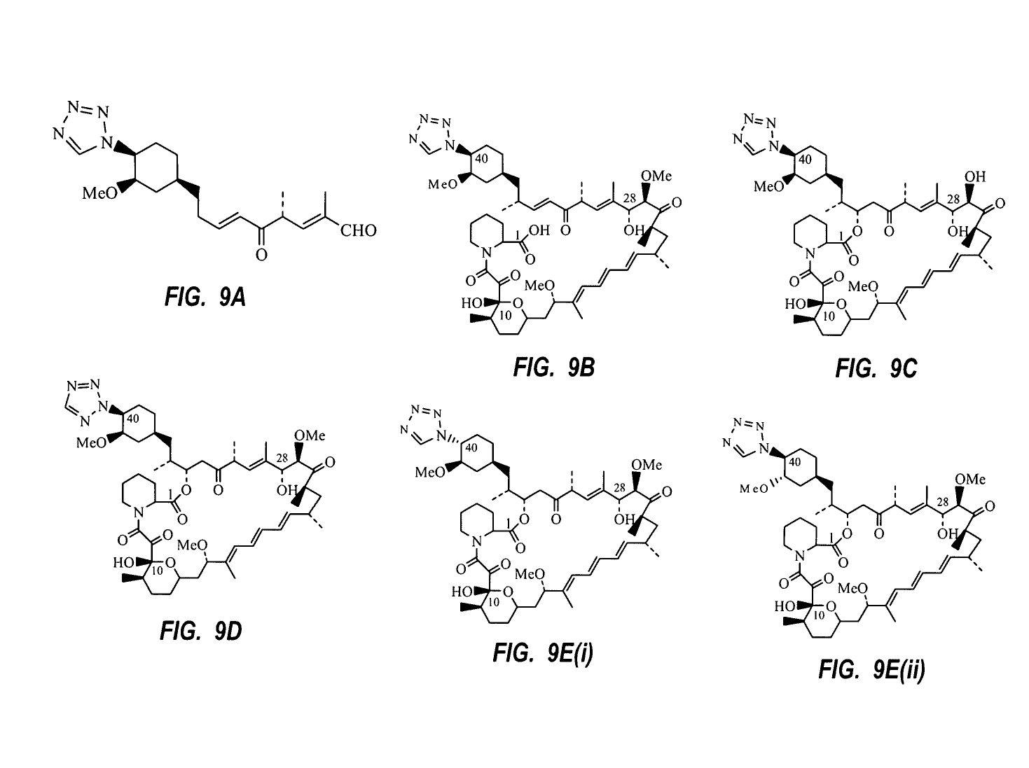

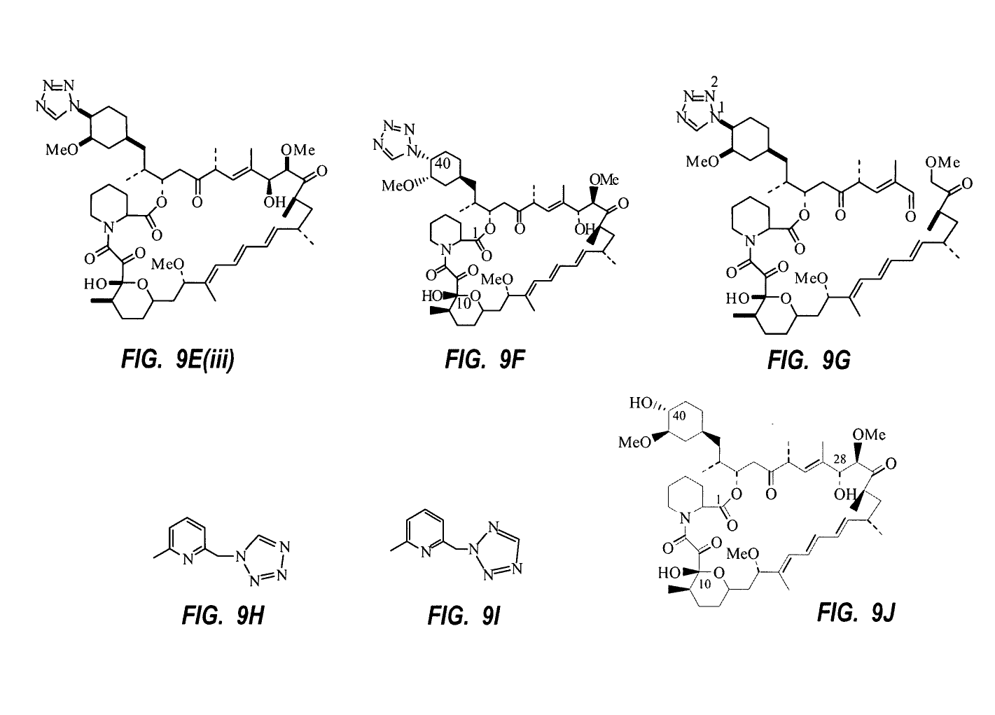

When the primary reaction product is ABT-578 (i.e., zotarolimus), the reaction impurities are separated therefrom. The reaction impurities are shown in FIGS. 9A-9I, and include the following: (FIG. 9A) aldehyde fragment of ABT-578; (FIG. 9B) N1 isomer open-ring acid of ABT-578; (FIG. 9C) des-methyl ABT-578; (FIG. 9D) N2 isomer of ABT-578, where the N2 isomer (FIG. 9D) may be purified as described herein as a primary product; (FIGS. 9E(i)-9E(iii)) three epimers of ABT-578; (FIG. 9F) di-epimers of ABT-578; (FIG. 9G) retro-aldol; and (FIG. 9H) N1-LTA (i.e., N1-Lutidine tetrazole adduc); (FIG. 9I) N2-LTA. Additionally, any unreacted rapamycin (FIG. 9J) is also separated from the reaction product ABT-578.

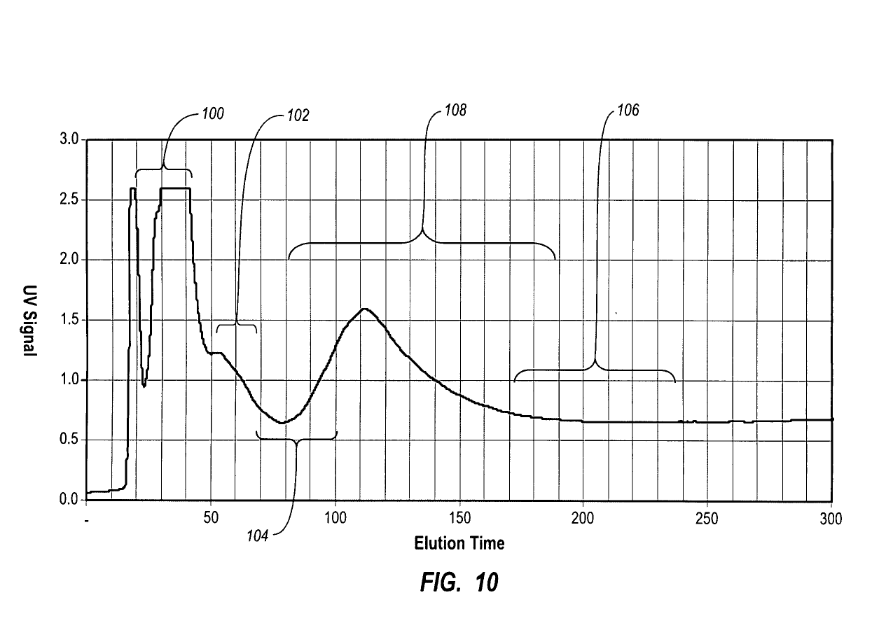

FIG. 10 shows a reaction product elution profile from a single column. As shown by the elution profile, the reaction impurities are separated from ABT-578 as follows: N2 isomer of ABT-578 (FIG. 9D) and N2-LTA (FIG. 9I) are shown to be present in the elutant at position 100; rapamycin (FIG. 9J) is shown to be present in the elutant at position 102; retro-aldol (FIG. 9G), N1 isomer open-ring acid of ABT-578 (FIG. 9B), des-methyl ABT-578 (FIG. 9C), three epimers of ABT-578 (FIGS. 9E(i)-9E(iii)), di-epimers of ABT-578 (FIG. 9F), and aldehyde fragment of ABT-578 (FIG. 9A) are shown to be present in the elutant at position 104; N1-LTA (FIG. 9H) is shown to be in the elutant at position 106; and the reaction product ABT-578 is shown to be present in the elutant at position 108. The reaction product ABT-578 can be separated from the rest of the impurities using the systems and methods of the present invention.

While not specifically shown, the N2 isomer can be a reaction product and separated from the other reaction impurities. In part, this is because the N2 isomer elutes at about 2 Bed Volume (BV), while the N1 isomer starts eluting at about 3.5 BV.

### Example 4

The reaction product of Example 2 is purified using a two column system with an isocratic solvent system where the elutant from the first column is condensed before being introduced into the second column. The reaction product mixture is loaded onto the first column, and eluted with 50% THF in heptane solvent. Each collected fraction is analyzed with TLC, based upon the TLC readings, composite of fractions with products are collected and analyzed by HPLC method. The composites with HPLC results passed the specifications are then combined and concentrated to dryness using rotary evaporator. Once the drying is completed, appropriate amount of dichloromethane was added in order to dissolve the solid residue containing the rapamycin analog. The product in dichloromethane was then loaded onto the second column, and eluted with 30% Acetone in heptane solvent. Again, each collected fraction is analyzed with TLC, Based upon the TLC readings, composite of fractions with products are collected and analyzed by HPLC method.

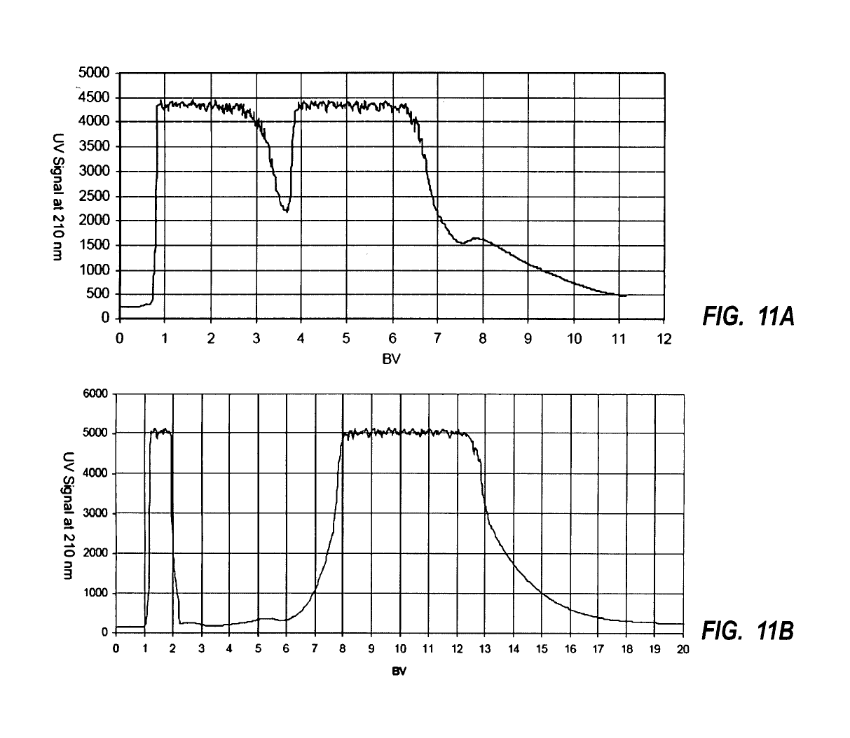

FIG. 11A is the typical elution profile for the first column (50% THF in heptane): The first major elution is product related impurities, while the 2^{nd} elution peak contains mainly product. FIG. 11B is the typical elution profile from the 2^{nd} column (30% Acetone in heptane). Table 1 is the summary of a typical recovery and purity results after the two columns purification with intermediate condensation of API (source: average of 20 runs), where PA is purity of the API at 97.7%. The main impurities are shown as Impurity #1 (retro-aldol) and Impurity #2 (N1-LTA).

TABLE 1

| | Two Columns W/Intermediate |
|---|---|
| | Condensation |
| Recovery (%) | 75.8% |
| ABT-578, PA % | 97.7% |
| Impurity #1 (retro-aldol) | 0.79% |
| Impurity #2 (N1-LTA) | 0.16% |

### Example 5

The reaction product of Example 2 is purified using a cascade system with isocratic elution solvent of 50% THF and heptane. Briefly, the reaction mixture is loaded onto the first column, and eluted with 50% THF in heptane solvent. As soon as the end of the first eluted peak is detected, the second column is brought into line (i.e., the outlet of the first column elution is switched from the waste line to the inlet of the second column). Each collected fraction is analyzed with TLC, Based upon the TLC readings, composite of fractions with products are collected and analyzed by HPLC method. The elutant is only concentrated after being obtained from the second column.

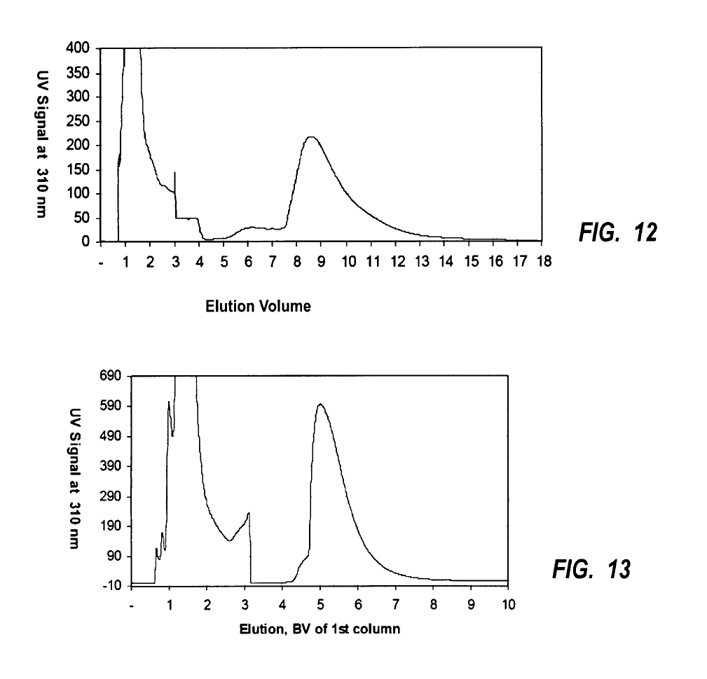

FIG. 12 is the typical elution profile of the elution profile via the cascade with isocratic elution with 50% THF in heptane. Table 2 is the summary of a typical recovery and purity results for the two column system of Example 4 compared to the cascade system of this example. It can be seen that the overall purity of the rapamycin analog substantially increases by using the isocratic cascade system that does not include a concentration step between columns. While impurity #1 slightly increases, the overall amount of impurities decreases.

TABLE 2

| | Two Columns | |
|---|---|---|
| | W/Condensation | Isocratic Cascade |
| Recovery (%) | 75.8% | 79.4% |
| ABT-578, PA % | 97.7% | 98.5% |
| Impurity #1 (retro-aldol) | 0.79% | 0.85% |
| Impurity #2 (N1-LTA) | 0.16% | None Detected |

It should be noted here that the level of a N1-LTA reaction byproduct has been removed completely by the isocratic cascade system. However, the other reaction byproduct, a retro-aldol, slightly increases from the isocratic cascade system. It has been found that this impurity is generated from the reaction between the product and the packed silica in the column. Therefore, the step gradient system is then evaluated to reduce the product decomposition (in the next example) by decreasing the residence time of the product in the column (i.e., reduce the contact time between product and silica).

### Example 6

The reaction product of Example 2 is purified using a cascade system with step gradient elution solvent. The step gradient solvent is as follows: 1) 50% THF and heptane stepping to 60% THF and heptane; and 2) 50% THF in heptane stepping to 30% acetone in heptane. The reaction mixture is loaded onto the first column, and eluted with 50% THF in heptane solvent. As soon as the first eluted peak ends, the second column is brought into line, and at the same time the elution solvent is stepped from 50% THF in heptane to 60% THF in heptane. Each collected fraction is analyzed with TLC. Based upon the TLC readings, composite of fractions with products are collected and analyzed by HPLC method. The elutant from the first column is not condensed or processed before being introduced into the second column. It should be noted that the system of 50% THF stepping to 30% acetone does not work, producing a poor quality of material. FIG. 13 is the typical elution profile with the cascade system using step gradient elution solvent. Table 3 is the summary of a typical recovery and purity results, which shows that the level of retro-aldol (impurity #1) decreases with the step gradient cascade system compared with the other systems.

TABLE 3

| | Isocratic Cascade | Step Gradient Cascade |
|---|---|---|
| Recovery (%) | 79.4% | 82.3% |
| ABT-578, PA % | 98.5% | 99.4% |
| Impurity #1 (retro-aldol) | 0.85% | 0.32% |
| Impurity #2 (N1-LTA) | None Detected | None Detected |

### Example 7

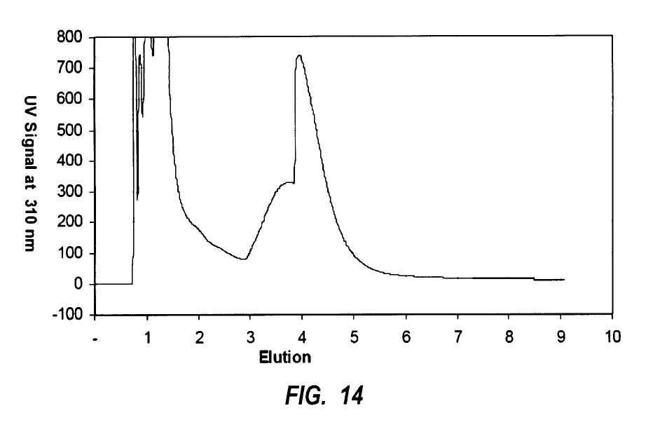

The reaction product of Example 2 is purified using a single column with step gradient solvent of 50% THF in heptane stepping to 60% THF in heptane. With the success of the cascade step gradient system, a step gradient system in a single column is evaluated. The reaction mixture is loaded onto the first column, and eluted with 50% THF in heptane solvent. As soon as the end of the first eluted peak is detected, the elution solvent is stepped from 50% THF in heptane to 60% THF in heptane. Each collected fraction is analyzed with TLC. Based upon the TLC readings, composite of fractions with products are collected and analyzed by HPLC method. FIG. 14 is the typical elution profile with step gradient using single column. Table 4 is the summary of a typical purity results for a single column with step gradient solvent of 50% THF in heptane stepping to 60% THF in heptane.

TABLE 4

| | |
|---|---|
| Recovery (%) | Not Measured |
| ABT-578, PA % | 97.8% |
| Impurity #1 (retro-aldol) | 0.66% |
| Impurity #2 (N1-LTA) | None Detected |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein in their entirety by specific reference.

What is claimed is:

1. A method of purifying an active pharmaceutical ingredient sufficient for administration into a human subject, the method comprising:
obtaining a reaction product composition having the active pharmaceutical ingredient and impurities, wherein said active pharmaceutical ingredient is rapamycin or a rapamycin analog;
introducing the reaction product composition into a first column of a chromatography system;
directing a first portion of a first elutant from the first column to waste;
directing a second portion of the first elutant from the first column into a second column without processing the second portion of the first elutant before entering the second column;
collecting factions of a second elutant from the second column that include the active pharmaceutical ingredient; and
concentrating said collected fractions to obtain a purity of the active pharmaceutical ingredient greater than 98% and with less than or about 0.95% being first and second major impurities.

2. A method as in claim 1, wherein the active pharmaceutical ingredient is a rapamycin analog.

3. A method as in claim 2, wherein the active pharmaceutical ingredient has a chemical structure of Formula 1 or derivative thereof:

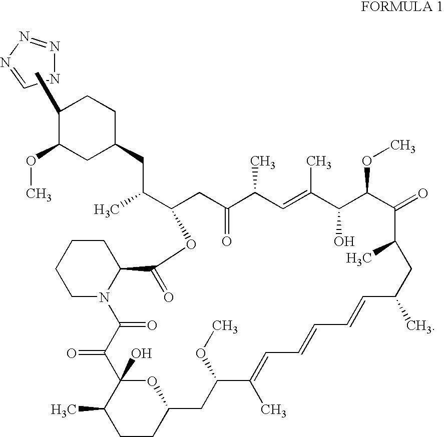

4. A method as in claim 3, wherein the active pharmaceutical ingredient has a chemical structure of Formula 2 or derivative thereof:

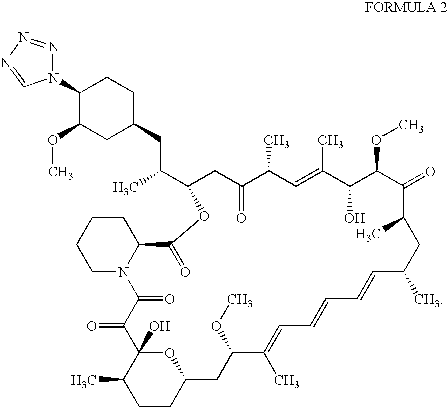

5. A method as in claim 3, wherein the first major impurity is a retro-aldol, and the second major impurity is a N1-lutidine tetrazole adduct.

6. A method as in claim 5, wherein the chromatography system is a cascade system with an isocratic solvent system.

7. A method as in claim 6, wherein the isocratic solvent system is THF and heptane.

8. A method as in claim 6, wherein the concentrated active pharmaceutical ingredient has a purity of greater than or about 98.5% and the first major impurity is less than or about 0.85% and the second major impurity is less than or about 0.1%.

9. A method as in claim 5, wherein the chromatography system is a cascade system with a step gradient solvent system.

10. A method as in claim 9, wherein the step gradient solvent system includes the following:
a first solvent introduced into the first column during a first time period between the introduction of the reaction product composition into a first column and the directing of the second portion of the first elutant from the first column into the second column; and
a second solvent introduced into the first column during a second time period after directing the second portion of the first elutant from the first column into the second column.

11. A method as in claim 10, wherein the first solvent includes a first ratio of THF/heptate and the second solvent includes a second ratio of THF/heptane, and wherein the first ratio is less than the second ratio.

12. A method as in claim 10, wherein the concentrated active pharmaceutical ingredient has a purity of greater than or about 99% and the first major impurity is less than or about 0.40% and the second major impurity is less than or about 0.1%.

13. A method of purifying a rapamycin analog sufficient for administration into a human subject, the method comprising:
obtaining a reaction product composition having the rapamycin analog and impurities;
introducing the reaction product composition into a first column of a cascade system with an isocratic solvent system;
directing a first portion of a first elutant from the first column to waste;
directing a second portion of the first elutant from the first column into a second column without processing the second portion of the first elutant before entering the second column;
collecting factions of a second elutant from the second column that include the active pharmaceutical ingredient; and
concentrating said collected fractions to obtain a purity of the active pharmaceutical ingredient greater than 98% and with less than or about 0.95% being first and second major impurities;
wherein said rapamycin analog has a chemical structure of Formula 1 or derivative thereof:

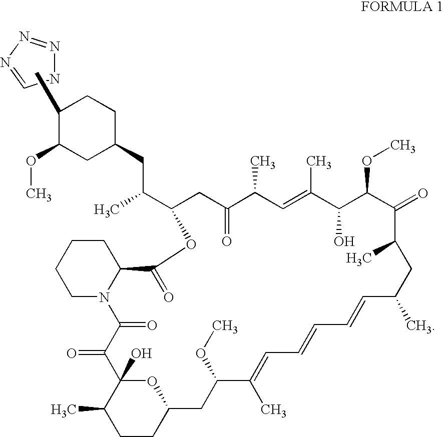

14. A method as in claim 13, wherein the active pharmaceutical ingredient has a chemical structure of Formula 2 or derivative thereof:

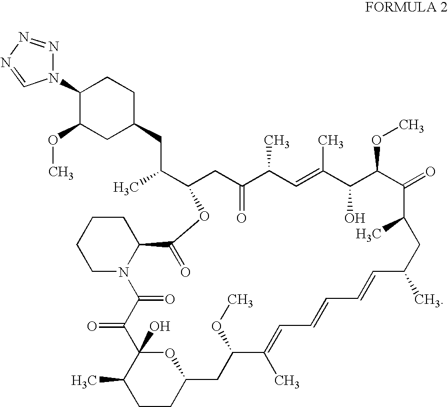

15. A method as in claim 13, wherein the first major impurity is a retro-aldol, and the second major impurity is a N1-lutidine tetrazole adduct.

16. A method as in claim 5, wherein the isocratic solvent system is THF and heptane.

17. A method as in claim 15, wherein the concentrated active pharmaceutical ingredient has a purity of greater than or about 98.5% and the first major impurity is less than or about 0.85% and the second major impurity is less than or about 0.1%.

18. A method of purifying a rapamycin analog sufficient for administration into a human subject, the method comprising:
obtaining a reaction product composition having the rapamycin analog and impurities;
introducing the reaction product composition into a first column of a cascade system with a step gradient solvent system;
directing a first portion of a first elutant from the first column to waste;
directing a second portion of the first elutant from the first column into a second column without processing the second portion of the first elutant before entering the second column;
collecting factions of a second elutant from the second column that include the active pharmaceutical ingredient; and
concentrating said collected fractions to obtain a purity of the active pharmaceutical ingredient greater than 98% and with less than or about 0.95% being first and second major impurities;
wherein said rapamycin analog has a chemical structure of Formula I or derivative thereof:

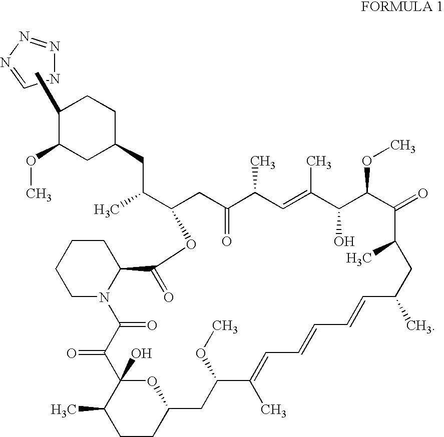

19. A method as in claim 18, wherein the active pharmaceutical ingredient has a chemical structure of Formula 2 or derivative thereof:

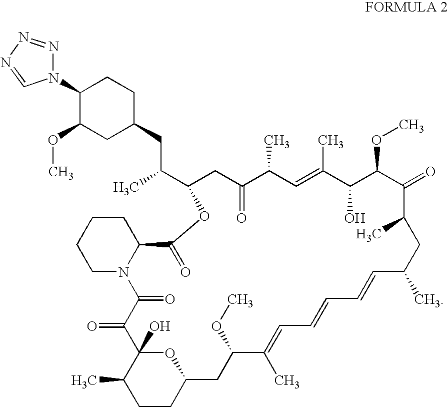

20. A method as in claim 18, wherein the first major impurity is a retro-aldol, and the second major impurity is a N1-lutidine tetrazole adduct.

21. A method as in claim 19, wherein the step gradient solvent system includes the following:
a first solvent introduced into the first column during a first time period between the introduction of the reaction product composition into a first column and the directing of the second portion of the first elutant from the first column into the second column; and
a second solvent introduced into the first column during a second time period after directing the second portion of the first elutant from the first column into the second column.

22. A method as in claim 21, wherein the first solvent includes a first ratio of THF/heptate and the second solvent includes a second ratio of THF/heptane, and wherein the first ratio is less than the second ratio.

23. A method as in claim 22, wherein the concentrated active pharmaceutical ingredient has a purity of greater than or about 99% and the first major impurity is less than or about 0.40% and the second major impurity is less than or about 0.11%.
